(19) United States
(12) Patent Application Publication
(10) Pub. No.: US 2026/0355072 A1
(43) Pub. Date: Nov. 12, 2026

(54) FOAM CONTAINING HYDROGEN GAS

(71) Applicant: VIAWA GMBH, Hennef (DE)

(72) Inventor: Markus POHLHAUSEN, Hennef (DE)

(73) Assignee: VIAWA GMBH, Hennef (DE)

(21) Appl. No.: 19/121,683

(22) PCT Filed: Oct. 17, 2023
(86) PCT No.: PCT/EP2023/078791

§ 371 Date: Apr. 16, 2025

(30) Foreign Application Priority Data
Oct. 17, 2022 (DE) 102022127153.6

(51) Int. Cl. A61K 8/04; A23L 2/54; A23L 29/10; A23P 30/40; A61K 8/19; A61K 8/365; A61K 8/55; A61K 8/73; A61K 8/9789; A61K 9/12; A61K 47/02; A61K 47/12; A61K 47/24; A61K 47/36; A61K 47/46; A61Q 19/00

(52) U.S. Cl. CPC A61K 8/046; A23L 2/54; A23L 29/10; A23P 30/40; A61K 8/19; A61K 8/365; A61K 8/553; A61K 8/73; A61K 8/9789; A61K 9/122; A61K 47/02; A61K 47/12; A61K 47/24; A61K 47/36; A61K 47/46; A61Q 19/00; A61K 2800/22

(57) ABSTRACT

A foam comprising a gas phase and an aqueous liquid phase, wherein the gas phase constitutes at least 50 vol.-% of the foam and essentially comprises hydrogen gas, and the aqueous liquid phase comprises at least one dissolved and/or dispersed emulsifier, and the liquid phase, diluted with 30 g of water per 0.1 g of liquid phase, has a surface tension at 20° C. of 40 to 90 mN/m. The foam can be safely and easily applied. A formulation for producing the foam contains at least one emulsifier which is soluble or dispersible in water. The foam can be used in a cosmetic product or as a cosmetic product, in a medical product or as a medical product, or in a food product or as a food product.

The present invention relates to a hydrogen gas-containing foam and a formulation for producing a hydrogen gas-containing foam, a method for producing the foam and the use of the foam.

Hydrogen gas exhibits an antioxidant and anti-inflammatory effect, which may be of interest in dermal applications in cosmetics, medicine or as a food additive or dietary supplement, for example. However, hydrogen gas is flammable even in relatively low concentrations of from 4 vol.-% in air, which makes its safe use a technological challenge. The volatility of the gas also poses practical challenges.

Three basic technological solutions are available to make volatile hydrogen accessible to consumers: finished products that already contain the hydrogen, hydrogen-producing electrical on-demand generators or chemical hydrogen generators/reservoirs that produce/release hydrogen immediately before or during use.

A particularly interesting type of application is hydrogen gas foam, which can contain large quantities of gas in its bubbles. Such a hydrogen gas foam should be dimensionally and volumetrically stable and non-flammable over the period of use and/or storage. The solutions known from the state of the art do not achieve these goals.

JP 2018/024613 A describes an industrially produced, finished cosmetic and its production, which is described as a “hydrogen gas-containing viscous composition”. The cosmetic is a finished gel or cream containing up to 70 vol.-% of hydrogen gas, which is produced by a plurality of manufacturing steps and hydrogen gas.

WO 2016/010139 A1 describes a cosmetic composition comprising a metal or a metal hydride powder mixed with a semi-fluid material such as a gel, a cream, a paste or a mousse. By bringing the powder into contact with moisture or a semi-fluid material, hydrogen gas is released or a composition containing hydrogen gas is obtained.

WO 2022/191757 A1 describes a composition and the production process of a foam for fire extinguishing, comprising a phospholipid or a hydrogenated phospholipid. The surface tension of the foam required for the application of extinguishing fire suitably lies below 18 mN/m.

KR 10-2021-0034530 describes a hydrogen gas-generating composition comprising a self-assembling peptide, a hydrogen compound, an acid and a carrier. It is stated that the self-assembling peptide generates a thin nano-film on the surface of an aqueous solution or on the surface of a solid carrier to maintain the hydrogen gas in the aqueous solution. US 2018/0092816 A describes a composition for dermal application comprising a hydrogen-generating compound or molecular hydrogen and a carrier containing a phosphatidylcholine component. It is explained that the phosphatidylcholine component encloses the hydrogen-generating compound or molecular hydrogen in the composition. Examples of products containing hydrogen gas include gels, creams, lotions and ointments. A foam or the materials required therefor are not discussed.

JP 2021/013376 A describes an industrially produced gel-like composition comprising 10 to 90 vol.-% of hydrogen and at least one thickener and an emulsifier. The gel-like composition is described as suitable for use in food or cosmetics and is produced by means of several manufacturing steps such as heating, technical auxiliaries and hydrogen gas.

WO 2018/131505 A1 describes an industrially produced ready-to-use gel and its production, which comprises 1 to 70 vol.-% of hydrogen gas and a thickening agent. The finished gel is intended for use as a cosmetic or foodstuff.

The problem underlying the present invention lies in providing hydrogen gas-containing foams which can be applied safely and easily.

The problem is solved by a foam comprising a gas phase and an aqueous liquid phase, wherein the gas phase constitutes at least 50 vol.-% of the foam and essentially comprises hydrogen gas, and the aqueous liquid phase comprises at least one dissolved and/or dispersed emulsifier, and the liquid phase, diluted with 30 g of water per 0.1 g of liquid phase, has a surface tension at 20° C. of 40 to 90 mN/m.

It was found that the surface tension of the liquid phase is of critical importance. While conventional surfactants greatly reduce the interfacial or surface tension and form surface films, the hydrogen gas foams formed are very liquid, run off quickly, are flammable and are not volumetrically stable. In contrast, hydrogen gas can be finely dispersed and forms bubbles with a small diameter in foams with a liquid phase according to the invention, wherein the foam is not flammable. In addition, the foam according to the invention is characterized by a particularly high form and volume stability, which is advantageous, for example, for dermal application.

The foam according to the invention is particularly fine-pored, i.e. has relatively small hydrogen gas bubbles. The average diameter of the gas bubbles is preferably at most 1.5 mm, particularly preferably at most 1.0 mm and most preferably at most 0.6 mm. The average diameter of the gas bubbles can be determined by means of light transmission, for example with a Krüss Dynamic Foam Analyzer DFA100.

In one embodiment, the reduction in foam volume within 60 min of the foam being provided is at most of 25 vol.-%, preferably at most of 20 vol.-%.

In a further embodiment, the reduction in foam volume within four weeks of the foam being provided is at most of 25 vol.-%, preferably at most of 20 vol.-%.

The gas phase of the foam constitutes at least 50 vol.-% of the foam and is dispersed in the aqueous liquid phase. Preferably, the gas phase constitutes at least 70 vol.-% of the foam, preferably at least 80 vol.-%, particularly preferably at least 90 vol.-%, at least 93 vol.-% or at least 95 vol.-%.

The gas phase essentially comprises hydrogen gas, preferably at least 60 vol.-%, preferably at least 90 vol.-%, most preferably at least 95 vol.-%, at least 98 vol.-% or at least 99 vol.-% of hydrogen gas.

The aqueous liquid phase comprises at least one emulsifier. The emulsifier and the other components of the aqueous liquid phase, if present, are water-soluble or at least water-dispersible. The term “water-soluble” is understood to mean a solubility in water at 20° C. of at least 0.1 g/L. The term “water-dispersible” is understood to mean that the component is dispersible in water.

The aqueous liquid phase is characterized by a specific surface tension. The surface tension can be determined using the Du Nouy ring method, e.g., according to the method described in the experimental section. The measurement is carried out on a diluted aqueous solution of the liquid phase. For this purpose, a sample of the liquid phase is placed in a volume of deionized water so that the liquid phase is diluted with 30 g of water per 0.1 g of liquid phase. For the practical implementation of the measurement, a measured quantity of the foam can also be used instead of the pure liquid phase. The liquid phase has a surface tension at 20° C. of 40 to 90 mN/m, preferably 50 to 90 mN/m, particularly preferably 60 to 80 mN/m.

In one embodiment, the individual dissolved or dispersed components of the liquid phase are each characterized in that an aqueous solution or dispersion of the component in the proportionate concentration in which the component is present in the liquid phase has a surface tension at 20° C. of at least 40 mN/m, preferably at least 50 mN/m and particularly preferably at least 60 mN/m, the aqueous solution or dispersion being diluted for measurement in an analogous manner with 30 g of water per 0.1 g of solution or dispersion.

The aqueous liquid phase preferably has a viscosity at 25° C. which is increased compared to water, preferably a viscosity of 100 mPa·s or more, preferably 100 to 20,000 mPa·s, more preferably 100 to 5,000 mPa·s, most preferably 100 to 3,500 mPa·s. The viscosity can be determined using a rotational rheometer according to the method described in the experimental section

The aqueous liquid phase comprises at least one emulsifier, i.e. a surface-active substance. Certain emulsifiers are capable of having a foam-stabilizing effect. The emulsifier can be selected from amphoteric, anionic, cationic and/or non-ionic emulsifiers.

In one embodiment, the emulsifier is selected from membrane-forming lipids, for example phospholipids, sphingolipids such as ceramides, glycolipids and sulfatides. Phospholipids such as lecithins are particularly noteworthy. Lecithins are mixtures of phosphoglycerides, comprising in particular phosphatidylcholine, phosphatidylethanolamine, phosphatidylinositol, phosphatidylserine and phosphatidic acid. Phosphatidylcholines are esters of fatty acids, glycerol, phosphoric acid and choline. Generally, phosphatidylcholines contain units of unsaturated fatty acids such as oleic acid or linolenic acid.

Lecithin can be produced synthetically or obtained technically, for example as an extract from egg yolk, soybeans, sunflowers, corn and/or rapeseed, preferably as an extract from sunflowers. In addition to the above-mentioned phosphoglycerides such as phosphatidylcholine, the technically obtained products include other impurities such as neutral fat from the extraction source. The term “lecithin” refers to the overall composition, which may also contain other phospholipids and neutral fat in addition to phosphatidylcholine and other phosphoglycerides.

In a preferred embodiment, the emulsifier is selected from hydrogenated membrane-forming lipids, in particular from hydrogenated lecithins.

Non-limiting examples are hydrogenated lecithin (CAS No.: 92128-87-5), hydrogenated phosphatidylcholine (CAS No.: 97281-48-6) or hydrogenated lysolecithin. These are obtained by hydrogenation of lecithin, phosphatidylcholine or lysolecithin, preferably from technically obtained lecithin, phosphatidylcholine or lysolecithin. Typically, the hydrogenation is carried out in the presence of a hydrogenation catalyst in an organic solvent.

Preferably, the hydrogenated lecithin comprises phosphatidylcholine in an amount of at least 60 wt.-%, preferably at least 75 wt.-%, more preferably at least 85 wt.-%, such as at least 90 wt.-% or at least 95 wt.-%. Hydrogenated lecithins are commercially available, for example Lipoid® P 100-3 or Phospholipon® 90 H (Lipoid) 90 H, Epikuron® 100 H or 200 SH (Cargill), Cosphaderm® SF-75-H or SB-75-H (Cosphatec), or Emulmetik® 320 (Lucas Meyer Cosmetics).

In a preferred embodiment, the aqueous liquid phase comprises hydrogenated lecithin in an amount of 3 to 100 wt.-%, based on the solids content of the aqueous liquid phase. Here and in the following, the term “solids content” refers to the total amount of dissolved or dispersed components of the aqueous liquid phase. In a particularly preferred embodiment, the aqueous liquid phase comprises hydrogenated lecithin in an amount of 100 wt.-%, based on the solids content of the aqueous liquid phase. In this case, hydrogenated lecithin is the only emulsifier.

In one embodiment, the emulsifier is selected from saponins. Saponins are glycosides of steroids, steroid alkaloids or triterpenes, and are widely distributed in higher plants. Saponins are found in high concentrations in chestnuts and in the bark of the South American soap bark tree, Quillaja saponaria.

Saponins can be produced synthetically or obtained technically, in particular as an extract from Quillaja saponaria. The term “saponins” refers to the overall composition, which may also contain other plant substances in addition to saponin.

In a preferred embodiment, the emulsifier is selected from saponins from Quillaja. Suitable saponins from Quillaja are commercially available, for example ANDEAN QDP ULTRA ORGANIC or ANDEAN QD (Desert King) or Foamex® Quillaja Extract Powder 60 (Garuda).

In a preferred embodiment, the aqueous liquid phase comprises saponins from Quillaja in an amount of 0.1 to 15 wt.-%, in particular 0.1 to 5 wt.-%, based on the solids content of the aqueous liquid phase.

In one embodiment, the emulsifier is selected from saponins and/or phosphoglycerides, preferably from saponins from Quillaja and/or hydrogenated lecithins.

The emulsifier usually has a considerable influence on the surface tension of the aqueous liquid phase. The influence on the surface tension can be concentration-dependent for some emulsifiers.

In one embodiment, the aqueous liquid phase further comprises a thickening agent. Thickening agents are substances that can bind water and increase the viscosity of a liquid phase by removing unbound water from it. Most thickening agents are linear or branched macromolecules, such as polysaccharides or proteins. It is possible for a substance to act as both an emulsifier and a thickening agent.

In one embodiment, the thickening agent is selected from polysaccharides. Polysaccharides are long-chain polymeric carbohydrates consisting of monosaccharide units linked together by glycosidic bonds. Examples of polysaccharides are glycogen, starch such as amylose and amylopectin, pectins, chitin, callose and cellulose.

Polysaccharides can be produced synthetically or obtained technically, for example as an extract from Tremella. Tremella is a fungus and belongs to the so-called tremblers, which form gelatinous to cartilaginous fruit bodies. A special feature of Tremella mushrooms is that their pharmacologically active polysaccharides make up the majority of the structural polysaccharides in the fruit body, whereas in other medicinal mushrooms the pharmacologically active polysaccharides only make up a small proportion of the biomass.

The main pharmacologically active substance of Tremella is the polysaccharide glucuronoxylomannan, consisting of a long α-mannose chain with various two- to three-membered oligosaccharide side chains of β-mannose, α-mannose, β-xylose and β-glucuronic acid, which are O-acetylated at the terminal mannoses. The chemical structure of Tremella glucuronoxylomannan varies in different samples of one species. The general ratio of xylose:glucuronic acid:mannose in Tremella fuciformis is provided as 1.0:2.77:4.9; 2:1:4 in T. aurantia and 7:1:5 in T. mesenterica, see Fraser et al., 1973, Can. J. Biochem. 51:219 to 224. Some additional saccharides could be identified in different samples of T. fuciformis, such as glucose, fucose, xylobiose and fructose.

Technically obtained polysaccharides include impurities in addition to polysaccharides. The term “polysaccharides” refers to the overall composition, which may also contain other plant compounds in addition to polysaccharides.

Suitable polysaccharides from Tremella are commercially available, for example Tremella fuciformis polysaccharide (REB Technology), Tremoist TP™ (Nippon), Phytochare-HA™ (applechem).

In a preferred embodiment, the aqueous liquid phase comprises polysaccharides from Tremella in an amount of 0.1 to 15 wt.-%, in particular 1 to 9 wt.-%, based on the solids content of the aqueous liquid phase.

In a particularly preferred embodiment, the aqueous liquid phase comprises an emulsifier selected from saponins, in particular saponins from Quillaja, and a thickening agent selected from polysaccharides, in particular polysaccharides from Tremella. Preferably, the aqueous liquid phase comprises saponins from Quillaja in an amount of 0.1 to 15 wt.-%, in particular 0.1 to 5 wt.-%, and polysaccharides from Tremella in an amount of 0.1 to 15 wt.-%, in particular 1 to 9 wt.-%, based on the solids content of the aqueous liquid phase.

The foams described here may comprise other components in addition to the ingredients described above. Particularly suitable are auxiliary substances that do not reduce the stability of the foam and maintain the surface tension and viscosity of the aqueous liquid phase discussed above. Also included are reaction products or incompletely reacted substances from chemical reactions that can release hydrogen gas.

Examples of suitable auxiliary agents include in particular particulate components which have a stabilizing effect in Pickering emulsions, for example microparticles such as spray-dried soy protein particles, hydrophobically modified starch particles and/or natural spore particles; and nanoparticles, such as cellulose nanocrystals, flavonoid (tiliroside) particles, chitin nanocrystals, ethylcellulose particles, chemically modified starch nanospheres, corn protein (zein) particles and/or solid lipid particles. Minerals (e.g., talc, mica, diatomaceous earth, illite) with a particle size of 10 nm to 300 μm are also suitable additives.

The auxiliary agents are preferably present in a total amount of 0 to 80 wt.-%, particularly preferably in a total amount of 0 to 40 wt.-%, based on the solids content of the aqueous liquid phase.

In one embodiment, the foam according to the invention additionally comprises a non-aqueous liquid phase which forms an emulsion together with the aqueous liquid phase. The non-aqueous liquid phase comprises, for example, an oil. The oil is preferably selected from polyol fatty acid polyesters and mineral oils, and mixtures thereof. The oil is particularly preferably selected from triglycerides of synthetic, semi-synthetic and natural origin, and mixtures thereof. Examples include triheptanoin, squalane, coconut oil such as MCT oil, olive oil, sunflower oil, soybean oil, peanut oil, rapeseed oil, almond oil, palm oil, castor oil, wheat germ oil, grapeseed oil, safflower oil, evening primrose oil, macadamia nut oil, corn oil, avocado oil and the like. In one embodiment, the foam according to the invention does not comprise a non-aqueous liquid phase.

The foams described here are understood to be liquid foams. Liquid foam comprises gaseous bubbles enclosed by liquid walls. The foam is preferably an aqueous foam. The foam is easy to spread and its consistency is comparable to that of shaving foam, for example.

The foam according to the invention is suitable, for example, for dermal application, i.e. for topical application to the skin. Preferred dermal applications include cosmetic applications and medical applications, in particular cosmetic applications. For example, the foam can be used to improve the appearance of the skin, such as to reduce skin irritations.

The invention also relates to the use of the foam according to the invention in a cosmetic product or as a cosmetic product, in a medical product or as a medical product, or in a foodstuff or as a foodstuff. The foam according to the invention can, for example, be added to creams, gels, mousses or beverages.

Furthermore, additives can be added to the foam according to the invention. Non-limiting examples are colorants, fragrances or flavorings, foodstuffs, food additives, cosmetic and pharmaceutical active ingredients.

The pH value of the foam according to the invention in a pH skin-neutral application is preferably in the range of 4.0 to 7.0, in particular 4.0 to 5.0, as determined at 20° C. under normal pressure; in a basic application, the pH value is above 7.0, but not higher than 10.35, as determined at 20° C. under normal pressure.

The invention also relates to a formulation for producing the hydrogen gas-containing foam according to the invention, comprising at least one water-soluble or water-dispersible emulsifier as described above.

The individual components of the formulation according to the invention can be liquid, gel-like or solid, for example in powder form. The individual components of the formulation according to the invention and thus the formulation itself can comprise water or be anhydrous.

The formulation according to the invention preferably comprises one or more pH regulators. The term “pH regulators” is understood to relate to compounds which change the pH value of aqueous liquid, i.e. both acids and bases. The pH regulator or regulators advantageously favor or accelerate the release of hydrogen gas.

The formulation preferably comprises 2 to 90 wt.-% of the pH regulator, preferably 10 to 85 wt.-%, particularly preferably 30 to 75 wt.-%, based on the total weight of the formulation.

The pH regulator is selected from acids, such as mineral acids, hydroxycarboxylic acids and amino acids, and bases, such as metal hydroxides.

Suitable mineral acids include sulfuric acid, nitric acid, phosphoric acid and/or hydrochloric acid.

Suitable hydroxycarboxylic acids include tartronic acid and fruit acids, such as citric acid, lactic acid, mandelic acid, tartaric acid and/or malic acid.

Suitable amino acids include glutamic acid.

Suitable metal hydroxides include sodium hydroxide and/or potassium hydroxide.

Preferably, the pH regulator comprises a fruit acid, such as citric acid, lactic acid, mandelic acid, tartaric acid or malic acid. In a particularly preferred embodiment, the pH regulator comprises citric acid.

The invention also relates to a foam according to the invention, produced by dispersing hydrogen gas in a formulation according to the invention. In this case, the formulation preferably contains water or water is added.

The invention also relates to a process for preparing a hydrogen gas-containing foam, comprising dispersing hydrogen gas in a formulation as described above in the presence of water. The water may be added to the formulation or may already be present in the formulation. The preparation of the foam may take place at below 40° C., preferably at ambient temperature such as 20° C.

Dispersing can be carried out under a hydrogen gas atmosphere and the hydrogen gas can be introduced into the aqueous formulation, similar to the production of cream, for example.

To disperse the hydrogen gas, hydrogen gas can also be introduced into the water-containing formulation with simultaneous mixing. A nozzle or a porous gas diffuser can be used for the introduction. For a sufficiently high power input, a stirring machine is generally used for mixing, for example a hand mixer, a disperser or a milk frother.

In one embodiment, the process is carried out under a hydrogen gas atmosphere and under the simultaneous introduction of hydrogen gas.

Electrical aids such as ultrasonic devices, dispersers and/or manual stirrers can be used in the process. The production of very small bubbles in the nano range is possible when using electrical aids.

It was found that the dispersion of hydrogen gas in the aqueous formulation allows the provision of a particularly creamy and very stable foam.

The foam according to the invention as produced by dispersing hydrogen gas can be stored until use in suitable packaging with minimal hydrogen gas permeability, such as an aluminum foil bag.

In a preferred embodiment, the formulation contains a compound that generates hydrogen gas upon contact with water. In this case, the formulation is preferably in powder form.

The formulation preferably comprises 0.5 to 20 wt.-% of the particulate compound which generates hydrogen gas with water, preferably 2 to 18 wt.-%, particularly preferably 3 to 15 wt.-%, based on the solids content of the formulation.

Preferably, the hydrogen gas generating compound is selected from a metallic powder, an alkali metal hydride, an alkaline earth metal hydride and/or a metal borohydride. The hydrogen gas-generating compound may be coated, for example with a polysaccharide such as pullulan.

Suitable metallic powders include alkali metals such as sodium and/or potassium; and alkaline earth metals such as magnesium and/or calcium.

Suitable alkali metal hydrides include lithium hydride (LiH), sodium hydride (NaH) and/or potassium hydride (KH).

Suitable alkaline earth metal hydrides include magnesium hydride (MgH₂), calcium hydride (CaH₂), barium hydride (BaH₂), beryllium hydride (BeH₂) and/or strontium hydride (SrH₂).

Suitable metal borohydrides include lithium borohydride (LiBH₄) and sodium borohydride (NaBH₄).

Preferably, the hydrogen gas-generating compound is selected from alkali metal hydrides, alkaline earth metal hydrides and/or metal borohydrides, in particular alkaline earth metal hydrides and/or metal borohydrides. In a preferred embodiment, the hydrogen gas-generating compound is selected from magnesium hydride and/or sodium borohydride, and is in particular magnesium hydride.

The hydrogen gas-generating compound is present in particulate form. Preferably, the hydrogen gas-generating compound has an average particle size in the range of 0.1 to 100 μm, preferably 0.1 to 30 μm, particularly preferably 0.1 to 20 μm. A particle size in these ranges allows the release of small hydrogen gas bubbles from the hydrogen gas-generating compound, and thus a fine-pored foam.

In a preferred embodiment, the formulation comprises:
a) 0.5 to 20 wt.-% of a compound which generates hydrogen gas with water;
b) 3 to 90 wt.-% of the emulsifier;
c) from 2 to 90 wt.-% of the pH regulator; and
c) to 100 wt.-% of optional ingredients;
in relation to the total weight of the formulation.

The desired amount of hydrogen gas can be adjusted by the amount of hydrogen-generating compound and/or the amount of the pH regulator.

The invention also relates to a process for preparing a hydrogen gas-containing foam, comprising mixing a formulation as described above with water.

The formulation can also be present as a multi-component system. The multi-component system may contain an aqueous component. The ingredients forming the aqueous liquid phase may be distributed as desired among the individual components, with the hydrogen-generating compound not being included in the aqueous component.

To produce a hydrogen gas-containing foam, the components of the multicomponent system are mixed, whereby additional water can be added. Hydrogen gas is generated by reaction of the aqueous parts of the multicomponent system with the hydrogen gas-generating compound, and a foam according to the invention is formed.

The foam can be produced in a simple way by mixing the formulation with water by hand, for example using a spoon. The thus obtained foam is stable over a normal application period, for example up to 75 min. The foam can be produced at below 40° C., preferably at ambient temperature such as 20° C.

The invention is illustrated by the following examples.

### EXAMPLES

The following measurements and examples were carried out at an ambient temperature of approximately 20° C., unless otherwise stated.

### Method 1: Determination of the Surface Tension

The following laboratory materials were used:
1) 1 aluminum ring−diameter (D=2×r) 60 mm−approx. 5 g with 3 threads with a hook for hanging onto a precision dynamometer
2) precision force gauge (0.1 N maximum tensile force)
3) laboratory lifting platform
4) beaker (600 mL)
5) tripod base, tripod rod and holder
6) distilled water

The aqueous liquid phase of a foam or the foam itself is used as the sample material. The sample material is diluted in the beaker with 30 g of water per 0.1 g of sample material and stirred in by hand. The beaker is placed on the laboratory lifting platform. The precision dynamometer is suspended from the tripod rod via a hook attached to a sleeve.

The aluminum ring is suspended from the dynamometer and placed above the beaker. The laboratory lifting platform is slowly raised until the ring is completely immersed in the water. The immersion force (F1) is taken from the precision force gauge. The laboratory lifting platform is slowly lowered until the liquid lamella breaks off. The force at the time of tearing (F2) is taken from the precision dynamometer.

The following formula is used to calculate the surface tension σ in mN/m:

σ=(F2−F1)/(4*π*r)

### Method 2: Determination of Viscosity

A rotational rheometer (also known as a rotational viscometer) is used. In a rotational rheometer, a body is rotated in the liquid by a motor. During rotation, the required torque is measured. The dynamic viscosity of the liquid can then be determined from this, as well as from the exact geometry of the rotating body used and the rotational speed. A DVNext Wells-Brookfield cone/plate rheometer was used as the measuring device The viscosity was determined at 25° C.

### Method 3: Inflammability

An approx. 20 mL sample of the foam to be tested is taken with a spatula and touched with the flame of an ignited lighter. The foam is considered to be “flammable” if it visually shows one of the following properties:
1) the foam ignites almost completely;
2) the foam ignites on its surface;
3) the foam partially ignites; or
4) several individual blisters become inflamed.

### Method 4: Foam Stability

A Kruss Dynamic Foam Analyzer-DFA100 was used to determine the foam stability.

2 g of a foamable powdered composition are placed in the measuring column of the DFA 100 and mixed with 6 g of water. The mixture is then stirred by hand for 20 s using a glass rod and then analyzed using the DFA100.
The development of the foam volume over time and the proportion of hydrogen gas were determined. The following parameters and conditions were used for the analysis:
software: ADVANCE 1.15
instrument: DFA100 +FSM
sample holder: SH4503
temperature: room temperature
column: CY4573 (40 mm prism short)
altitude lighting: blue LED, 14%
structural lighting: 20%
camera height: 55 mm
camera position: ●●
measuring frequency: 0.5 fps (height); 0.1 fps (structure)

### Inventive Example 1 -1

6 g of hydrogenated lecithin (Phospholipon® 80 H, Lipoid) was added as a powder to 40 g of distilled water (proportion of hydrogenated lecithin in the liquid phase: 13 wt.-%) and whipped under a hydrogen gas atmosphere at room temperature using an electric milk frother. The resulting fine-pored foam was creamy, stable over the test period of 24 hours and non-flammable.

The following properties were determined:
surface tension: 74 mN/m
viscosity: greater than 100 mPa·s
hydrogen gas in the foam: 77 vol.-%
foam volume immediately after production: 200 mL
foam volume after 60 min: 200 mL
foam volume after 24 h: 195.5 mL (reduction of 2.25%)

### Inventive Example 1-2

0.1 g magnesium hydride (MgH2, Zegen Metals & Chemicals), 3 g hydrogenated lecithin (Cosphaderm SF-75H, Cosphatec) and 0.6 g citric acid (Citric Acid Monohydrate, Jungbunzlauer) were mixed.

The powder mixture was placed in a bowl and stirred quickly by hand with 10 g of distilled water using a spatula at room temperature for about 20 seconds, producing a fine-pored foam.

The foam was tested for its flammability and stability. It was found that the resulting foam was stable and non-flammable for the application time of a face mask of 30 minutes.

The following properties were determined:
surface tension: 74 mN/m
viscosity: greater than 100 mPa·s
hydrogen gas in the foam: 92 vol.-%
foam volume immediately after production: 180 mL
foam volume after 60 min: 150 mL (reduction of 16.7%)

### Inventive Example 1-3

0.1 g magnesium hydride (MgH₂, Zegen Metals & Chemicals), 0.04 g Quillaja wood extract (ANDEAN QDP ULTRA, Desert King), 0.07 g Tremella fuciformis Polysaccharide (Tremoist TP, Nippon) and 0.6 g citric acid (Citric Acid Monohydrate, Jungbunzlauer) were mixed.

The powder mixture was placed in a bowl and stirred quickly by hand with 10 g of distilled water using a spatula at room temperature for about 20 seconds, producing a fine-pored foam.

The foam was tested for its flammability and stability. It was found that the resulting foam was stable and non-flammable for the application time of a face mask of 30 minutes.

The following properties were determined:
surface tension: 72 mN/m
viscosity: greater than 100 mPa·s
hydrogen gas in the foam: 94 vol.-%
foam volume immediately after production: 180 mL
foam volume after 60 min: 150 mL (reduction of 16.7%)

### Inventive Example 1-4

0.1 g magnesium hydride (MgH₂, Zegen Metals & Chemicals), 0.04 g Quillaja wood extract (ANDEAN QDP ULTRA, Desert King), 1 g hydrogenated lecithin (Cosphaderm SF-75H, Cosphatec), 0.07 g Tremella fuciformis Polysaccharide (Tremoist TP, Nippon) and 0.6 g citric acid (Citric Acid Monohydrate, Jungbunzlauer) were mixed.

The powder mixture was placed in a bowl and stirred quickly by hand with 10 g of distilled water using a spatula at room temperature for about 20 seconds, producing a fine-pored foam.

The foam was tested for its flammability and stability. It was found that the resulting foam was stable and non-flammable for the application time of a face mask of 30 minutes.

The following properties were determined:
surface tension: 74 mN/m
viscosity: greater than 100 mPa·s
hydrogen gas in the foam: 93 vol.-%
foam volume immediately after production: 180 mL
foam volume after 60 min: 160 mL (reduction of 11.1%)

### Practical Example: Dermal Application

A foam according to the invention, as produced according to examples 1 -1 to 1-5 below, is applied to the skin previously cleansed, for example the face (approx. 200 ml of foam produced per facial application). The composition acts over a period of, for example, 10 to 30 min and is then rinsed off with water.

The applied foam is stable and non-inflammatory over the application time. It also remains stable on the face and does not run off. The foam “collapses” towards the skin and releases a lot of hydrogen gas and moisture into the skin.

### Comparative Example

0.1 g magnesium hydride (MgH2, Zegen Metals & Chemicals), 0.6 g citric acid (Citric Acid Monohydrate, Jungbunzlauer), 2.3 g sodium lauryl glucoside hydroxypropyl sulfonate and silica gel (SugaNate 160 Dry, ColonialChemie) were mixed.

The powder mixture was placed in a bowl and stirred quickly by hand with 10 g of distilled water using a spatula at room temperature for about 20 seconds to obtain a foam.

The foam was tested for flammability and stability. It was found that the resulting foam was flammable immediately after production (“flammable”: item 4).

The following properties were determined:
surface tension: 37 mN/m
viscosity: greater than 100 mPa·s
hydrogen gas in the foam: 78 vol.-%
foam volume immediately after production: 60 mL
foam volume after 60 min: 40 mL (reduction of 33.3%)

### Comparative Example 2 -2

0.1 g magnesium hydride (MgH2, Zegen Metals & Chemicals), 0.6 g citric acid (Citric Acid Monohydrate, Jungbunzlauer) and 2.3 g sodium cocoyl isethionate (HOSTAPON SCI 85 P, Clariant) were mixed.

The powder mixture was placed in a bowl and stirred quickly by hand with 10 g of distilled water using a spatula at room temperature for about 20 seconds to obtain a foam.

The foam was tested for flammability and stability. It was found that the resulting foam was flammable immediately after production (“flammable/flammable foam”: point 4).

The following properties were determined:
surface tension: 34 mN/m
viscosity: greater than 100 mPa·s
hydrogen gas in the foam: 74 vol.-%
foam volume immediately after production: 50 mL
foam volume after 60 min: 30 mL (reduction of 40%)

### Comparative Example 2-3

0.1 g magnesium hydride (MgH2, Zegen Metals & Chemicals), 0.6 g citric acid (Citric Acid Monohydrate, Jungbunzlauer) and 2.3 g sodium lauryl sulfoacetate were mixed.

The powder mixture was placed in a bowl and stirred quickly by hand with 10 g of distilled water using a spatula at room temperature for about 20 seconds to obtain a foam.

The foam was tested for its flammability and stability. It was found that the resulting foam was flammable immediately after production (“flammable”: item 4).

The following properties were determined:
surface tension: 30 mN/m
viscosity: greater than 100 mPa·s
hydrogen gas in the foam: 78 vol.-%
foam volume immediately after production: 60 mL
foam volume after 60 min: 30 mL (reduction of 50%)

### Comparative Example 2-4

0.1 g magnesium hydride (MgH2, Zegen Metals & Chemicals), 0.6 g citric acid (Citric Acid Monohydrate, Jungbunzlauer) and 2.3 g sucrose cocoate (TEGOSOFT LSE 65 K SOFT, Evonik) were mixed.

The powder mixture was placed in a bowl and stirred quickly by hand with 10 g of distilled water using a spatula at room temperature for about 20 seconds to obtain a foam.

The foam was tested for its flammability and stability. It was found that the resulting foam was flammable immediately after production (“flammable”: item 1).

The following properties were determined:
surface tension: 18 mN/m
viscosity: greater than 100 mPa·s
hydrogen gas in the foam: 87 vol.-%
foam volume immediately after production: 100 ml
foam volume after 60 min: 0 mL (completely decomposed)

It is evident that the foam stability is strongly dependent on the surface tension of the aqueous liquid phase. The foams according to the invention show high stability and are not flammable.

Comparative Examples 2-1 to 2-3 produced a foam with an inhomogeneous bubble size distribution and in some cases relatively large bubbles with a visually determined size of 2 mm or more. It has been shown that such hydrogen gas foams are flammable. Comparative Example 2-4 produced an initially fine-pored foam, which however quickly coalesced to bubble sizes of 3 mm or more. This foam ignited almost completely.

1. A foam comprising a gas phase and an aqueous liquid phase, wherein the gas phase constitutes at least 50 vol.-% of the foam and essentially comprises hydrogen gas, and the aqueous liquid phase comprises at least one dissolved and/or dispersed emulsifier, and the liquid phase, diluted with 30 g of water per 0.1 g of liquid phase, has a surface tension at 20° C. of 40 to 90 mN/m.

2. The foam according to claim 1, wherein the average diameter of the gas bubbles is at most 1.5 mm.

3. The foam according to claim 1, wherein the reduction in the foam volume within 60 min of the foam being provided is at most 25 vol.-%.

4. The foam according to claim 1, wherein the reduction in the foam volume within four weeks of the foam being provided is at most 25 vol.-%.

5. The foam according to claim 1, wherein the liquid phase has a viscosity at 25° C. which is increased compared to water.

6. The foam according to claim 1, wherein the emulsifier is selected from saponins and/or phosphoglycerides.

7. The foam according to claim 1, wherein the aqueous liquid phase further comprises a thickening agent.

8. A formulation for producing a hydrogen gas-containing foam according to claim 1, comprising at least one water-soluble or water-dispersible emulsifier.

9. The formulation according to claim 8, wherein the emulsifier is selected from saponins and/or phosphoglycerides.

10. The formulation according to claim 8, further comprising a thickening agent.

11. The formulation according to claim 8, further comprising a pH regulator selected from mineral acids, hydroxycarboxylic acids, amino acids and metal hydroxides.

12. The formulation according to claim 11, wherein the pH regulator is a hydroxycarboxylic acid wherein the hydroxycarboxylic acid is a fruit acid selected from citric acid, lactic acid, mandelic acid, tartaric acid or malic acid.

13. The formulation according to claim 8 for producing a hydrogen gas-containing foam by mixing with water, comprising a compound which generates hydrogen gas upon contact with water.

14. The formulation according to claim 13, wherein the formulation is in powder form.

15. The formulation according to claim 13, wherein the compound which generates hydrogen gas is selected from a metallic powder, an alkali metal hydride, an alkaline earth metal hydride and/or a metal borohydride.

16. The formulation according to claim 15, wherein the compound which generates hydrogen gas is sodium borohydride or magnesium hydride.

17. The formulation according to claim 13, comprising:
a) 0.5 to 20 wt.-% of the compound generating hydrogen gas on contact with water;
b) 3 to 90 wt.-% of the emulsifier;
c) from 2 to 90 wt.-% of the pH regulator; and
c) to 100 wt.-% of optional ingredients;
relative to the total weight of the formulation.

18. A process for preparing a hydrogen gas-containing foam, comprising dispersing hydrogen gas in a formulation according to claim 8.

19. A method of preparing a cosmetic product, medical product, or foodstuff comprising the step of adding the foam of claim 1 to the cosmetic product, medical product, or foodstuff.

20. The method of claim 19, comprising adding the foam to a cream, a gel, a mousse, or a beverage.
